(19) United States
(12) Patent Application Publication
(10) Pub. No.: US 2007/0244565 A1
(43) Pub. Date: Oct. 18, 2007

(54) Prosthetic humeral device and method

(76) Inventor: Robert P. Stchur, Punta Gorda, FL (US)

Correspondence Address: EDWARD M. LIVINGSTON, PA, 963 TRAIL TERRACE DRIVE, NAPLES, FL, 34103, US

(21) Appl. No.: 11/405,338

(22) Filed: Apr. 17, 2006

(51) Int. Cl. A61F 2/40

(52) U.S. Cl. 623/19.14; 623/23.27

(57) ABSTRACT

A prosthetic humeral device (1) having at least two guide holes (11) located at predetermined locations thereon for the acceptance of a fastening means (29), which is preferably a screw (16) and a predeterminedly-shaped washer (6) with at least one projection (17) located on the underside. After inserting the elongated stem (14) of the prosthetic humeral device (1) into the humerus (2), a tuberosity grasping tool (19) is used to grasp a tuberosity (4) and (7). The tuberosity (4) and (7) is then positioned to an appropriate level on the prosthetic humeral device (1), a hole is drilled into the tuberosity (4) and (7), the screw (16) with washer (6) is inserted, the grasping tool (19) is removed and the screw (16) and washer (6) are tightened.

### BACKGROUND OF THE INVENTION

This invention relates to prosthetic humeral devices, more particularly, a prosthetic humeral device that employs a threaded fastening means, rather than conventional sutures, to secure the tuberosities in place. A special tool is also provided to assist in the process.

As with any medical procedure wherein a patient undergoes surgery, there is always concern relating to contamination of an open wound and the amount of time the open wound is exposed as the longer a wound is open and exposed to the air, the more likely it is that the wound will become contaminated with air born matter. Thus, time is of the essence when operating on a patient.

Currently, hemi-shoulder replacements, also known as partial shoulder replacements, are surgeries commonly required for proximal humerus fractures or rotator cuff tears wherein a prosthetic humeral is placed within the humerus and attached to the socket via tuberosities and tendons. Conventional hemi-shoulder replacement prosthetic devices have fins located thereon with a plurality of holes located in the fins. The medical procedure for inserting and securing the humeral prosthetic usually takes approximately one and a half hours as a surgeon must mobilize the chunks of bone that have become broken, remove the head and tuberosities on the humerus bone, drill down into the canal of the humerus for insertion of the prosthetic humeral, insert the prosthetic humeral into the canal, attach a metal head onto the stem of humerus and insert approximately seven to nine sutures to affix the tendons, the greater tuberosity and the lesser tuberosity to the holes on the fins of the prosthetic humeral device. Although the amount of time to perform a hemi-shoulder replacement may seem minimal, approximately half of the time is spent on suturing the tuberosities and tendons to the prosthetic.

In addition, as there are a plurality of sutures that are required in order to secure the tuberosities and tendons to the prosthetic, oftentimes the sutures become entangled, thereby making the procedure more complicated and time-consuming.

Furthermore, because the greater tuberosity is sutured to the fin, it is very difficult to control the height of the greater tuberosity.

Also, as the patient is under anesthesia during the entire operation, there is an increased risk to the patient as a greater and more potent amount of anesthesia must be administered.

Finally, traditional suture fixation of the tuberosity fragments is often inadequate and prone to failure.

Therefore, due to these inefficiencies related to the suturing of the tuberosities and tendons to the prosthetic humeral, surgeons spend unneeded time and energy on this procedure and, more importantly, patients are exposed to an unnecessary increased risk of contamination and infection.

Thus, a need exists for a prosthetic humeral device and method that reduces the amount of time required to perform the procedure and to better secure the tuberosity fragments.

The relevant prior art includes the following references:

| Patent No. | | |
|---|---|---|
| (U.S. unless stated otherwise) | Inventor | Issue/Publication Date |
| U.S. Pat. No. 5,776,194 | Mikol et al. | Jul. 07, 1998 |
| EP 1479350 | Mikol et al. | Nov. 24, 2004 |
| U.S. Pat. No. 5,480,402 | Kim | Jan. 02, 1996 |
| U.S. Pat. No. 6,383,187 | Tormala et al. | May 07, 2002 |
| U.S. Pat. No. 6,520,994 | Nogarin | Feb. 18, 2003 |
| U.S. Pat. No. 6,406,496 | Ruter | Jun. 18, 2002 |
| U.S. Pat. No. 4,693,723 | Gabard | Sep. 15, 1987 |
| U.S. Pat. No. 5,489,309 | Lackey et al. | Feb. 06, 1996 |
| U.S. Pat. No. 6,558,425 | Rockwood, Jr. | May 06, 2003 |
| 2001/0037152 | Rockwood, Jr. | Nov. 01, 2001 |
| 2005/0090902 | Masini | Apr. 28, 2005 |

### SUMMARY OF THE INVENTION

The primary object of the present invention is to provide a prosthetic humeral device and method that reduces the amount of time to perform a hemi-shoulder replacement by at least half and to provide a more secure and accurate reduction of tuberosity fragments.

A further object of the present invention is to provide a prosthetic humeral device and method that eliminates the need for sutures.

An even further object of the present invention is to provide a prosthetic humeral device and method that provides a more secure fixation.

Another object of the present invention is to provide a prosthetic humeral device and method that reduces the amount of time the wound is exposed to the air, thereby reducing the risk of contamination and infection.

A further object of the present invention is to provide a prosthetic humeral device and method that permits greater control of the height of the greater tuberosity.

An even further object of the present invention is to provide a prosthetic humeral device and method that reduces the amount and intensity of anesthesia administered to the patient due to the reduced amount of time required for the procedure.

Another object of the present invention is to provide a prosthetic humeral device and method that promotes bone ingrowth of the greater and lesser tuberosities.

The present invention fulfills the above and other objects by providing a prosthetic humeral device comprised of a prosthetic humeral having at least two holes located at predetermined locations thereon for the acceptance of a fastening means. The fastening means is preferably a screw having a predeterminedly-shaped washer.

To use the prosthetic humeral device of the present invention, a surgeon performs essentially the same procedure as when inserting a conventional prosthetic humeral. However, rather than suturing the tuberosities and tendons to the holes in the fins of the conventional prosthetic humeral, the surgeon utilizes a tuberosity grasping tool to grasp a tuberosity, places the tuberosity to an appropriate level on the prosthetic humeral, inserts a circle guide into the corresponding threaded hole, inserts a screw that is located in the washer, removes the grasping tool and tightens the screw and washer wherein at least one projection located on the underside of the washer grabs both the humerus bone and tendon.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is an anterior plan view of the prosthetic humeral device of the present invention installed in a left humerus;

FIG. 2 is a posterior plan view of the prosthetic humeral device of the present invention installed in a left humerus;

FIG. 3 is a perspective view of the prosthetic humeral device of the present invention for a right humerus;

FIG. 4 is a perspective view of a first embodiment of a fastening means of the present invention;

FIG. 5 is a top view of a first embodiment of a fastening means of the present invention;

FIG. 6 is a perspective view of a circular plate of the present invention;

FIG. 7 is a perspective view of a grasping tool used in the installation of the prosthetic humeral device of the present invention;

FIG. 8 is an upward looking view of top circle guide of the grasping tool;

FIG. 9 is an upward looking view of a bottom circle guide of the grasping tool; and

FIG. 10 is a perspective view of a second embodiment of a fastening means of the present invention.

### DESCRIPTION OF THE PREFERRED EMBODIMENTS

For purposes of describing the preferred embodiment, the terminology used in reference to the numbered components in the drawings is as follows:

| | |
|---|---|
| 1. | prosthetic humeral device |
| 2. | humerus |
| 3. | scapula |
| 4. | lesser tuberosity |
| 5. | subscapularis |
| 6. | washer |
| 7. | greater tuberosity |
| 8. | supia spinatus |
| 9. | infra spinatus |
| 10. | teres minor |
| 11. | guide holes |
| 12. | lateral suture hole |
| 13. | medial suture hole |
| 14. | elongated stem |
| 15. | screw head |
| 16. | screw |
| 17. | projection |
| 18. | guiding hole area |
| 19. | grasping tool |
| 20. | locking mechanism |
| 21. | bottom circle guide |
| 22. | drill guide projection |
| 23. | handle |
| 24. | elevated drill guide |
| 25. | slot |
| 26. | spike |
| 27. | top circle guide |
| 28. | prong |
| 29. | fastening means |
| 30. | hole for screw |
| 31. | crook |
| 32. | humeral head |
| 33. | underside of washer |

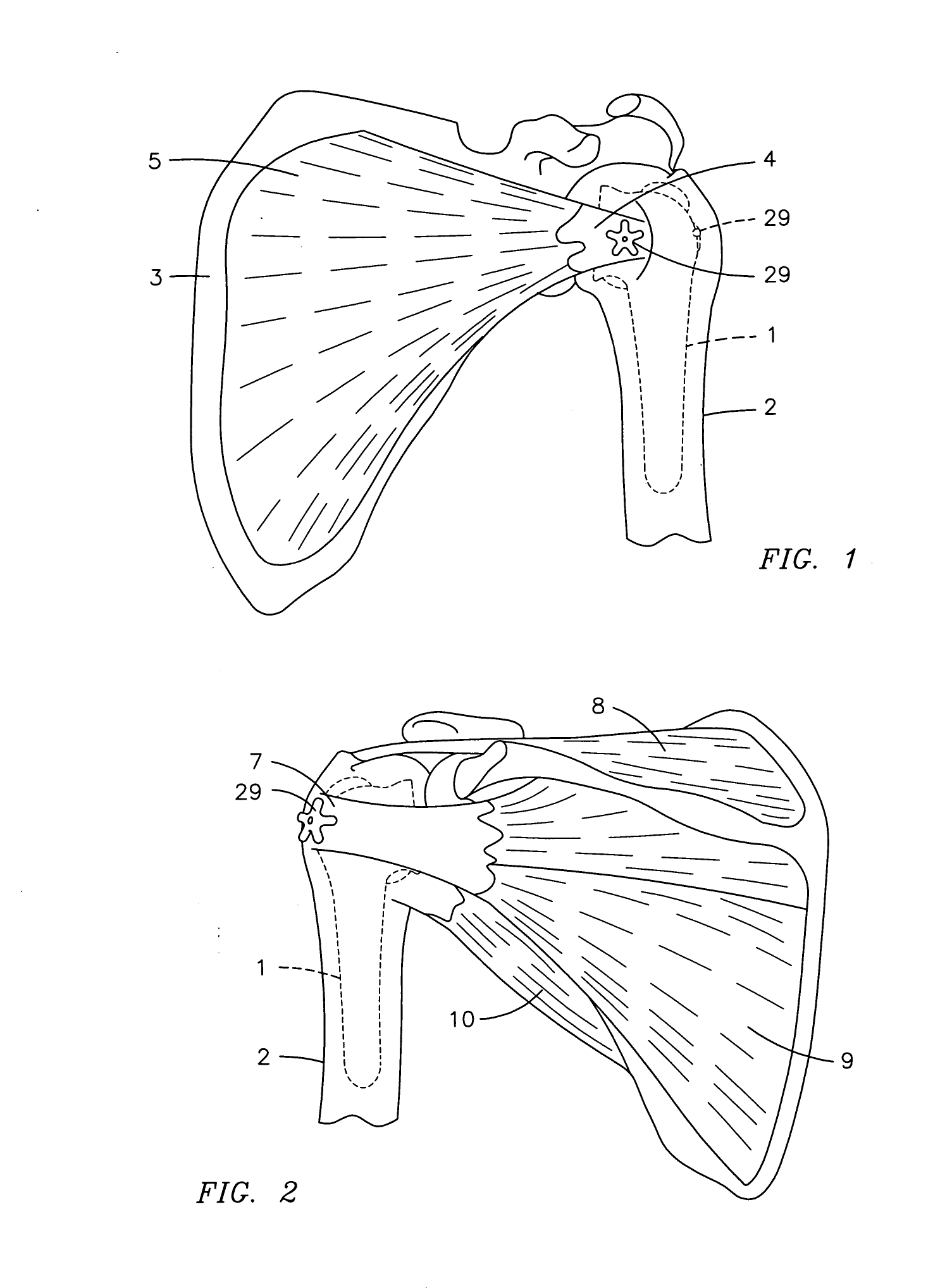

With reference to FIG. 1, an anterior plan view of the prosthetic humeral device of the present invention installed in a left humerus is shown. The prosthetic humeral device 1 is installed within the canal of a humerus 2 via the method described below. The lesser subscapularis 5, which is attached to the scapula 3, has a lesser tuberosity 4 that is secured to the prosthetic humeral device 1 via a fastening means 29.

FIG. 2 shows a rear or posterior plan view of the prosthetic humeral device of the present invention installed in a left humerus. The muscles of the rotator cuff located on the back include the supia spinatus 8, the infra spinatus 9 and the teres minor 10. When the prosthetic humeral device 1 of the present invention is installed in the canal of the humerus 2, the greater tuberosity 7 is secured to the prosthetic humeral device 1 via a fastening means 29.

In FIG. 3, a perspective view of the prosthetic humeral device of the present invention for a right humerus is shown. The prosthetic humeral device 1 includes an elongated stem 14 that fits within the canal of a humerus 2, a humeral head 32 and at least two guiding hole areas 18 wherein each guiding hole area 18 has a centrally-located, internally-threaded guiding hole 11. The prosthetic humeral device 1 may include optional medial suture holes 13 and a lateral suture hole 12 in the humeral head 32.

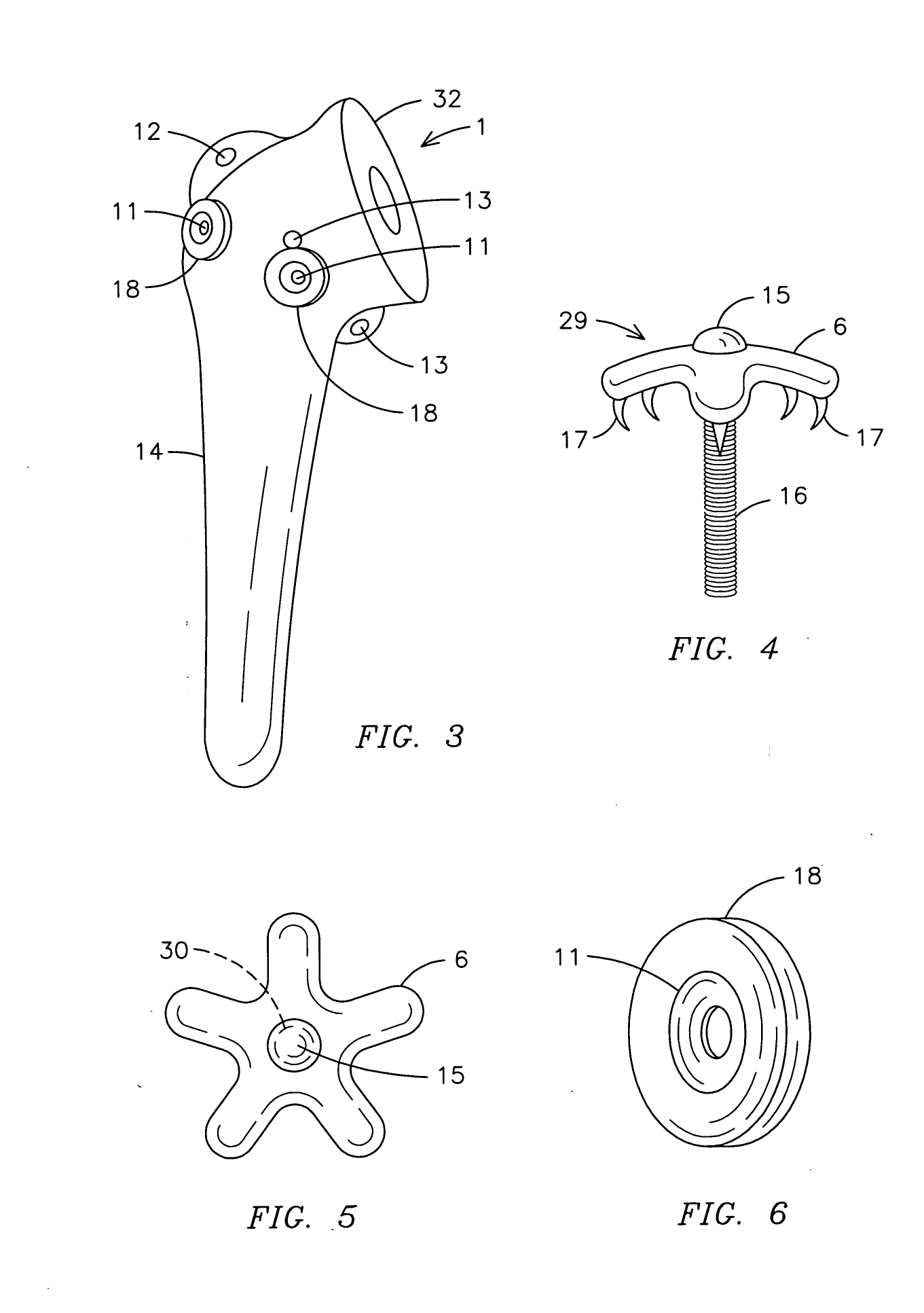

With reference to FIG. 4, a perspective view of a first embodiment of a fastening means of the present invention is shown. The fastening means 29 includes a screw 16 having a screw head 15 that is inserted through a washer 6. Located on an underside of the washer 33 is at least one projection 17 that secures the washer 6 to the humerus 2 and tendons. The washer 6 is preferably convex so as to provide a more conforming securement of the tuberosities to the humerus 2 and prosthetic humeral device 1.

In FIG. 5, a top view of a first embodiment of a fastening means of the present invention is shown. The washer 6 is preferably star-shaped and has a centrally located hole 30 for the insertion of a screw 16.

FIG. 6 shows a perspective view of a guiding hole area of the present invention. The guiding hole area 18 includes a guide hole 11 for the insertion of a screw 16. The guide hole 11 narrows so as the diameter of the guide hole 11 becomes smaller to assist in the guidance of the screw 16.

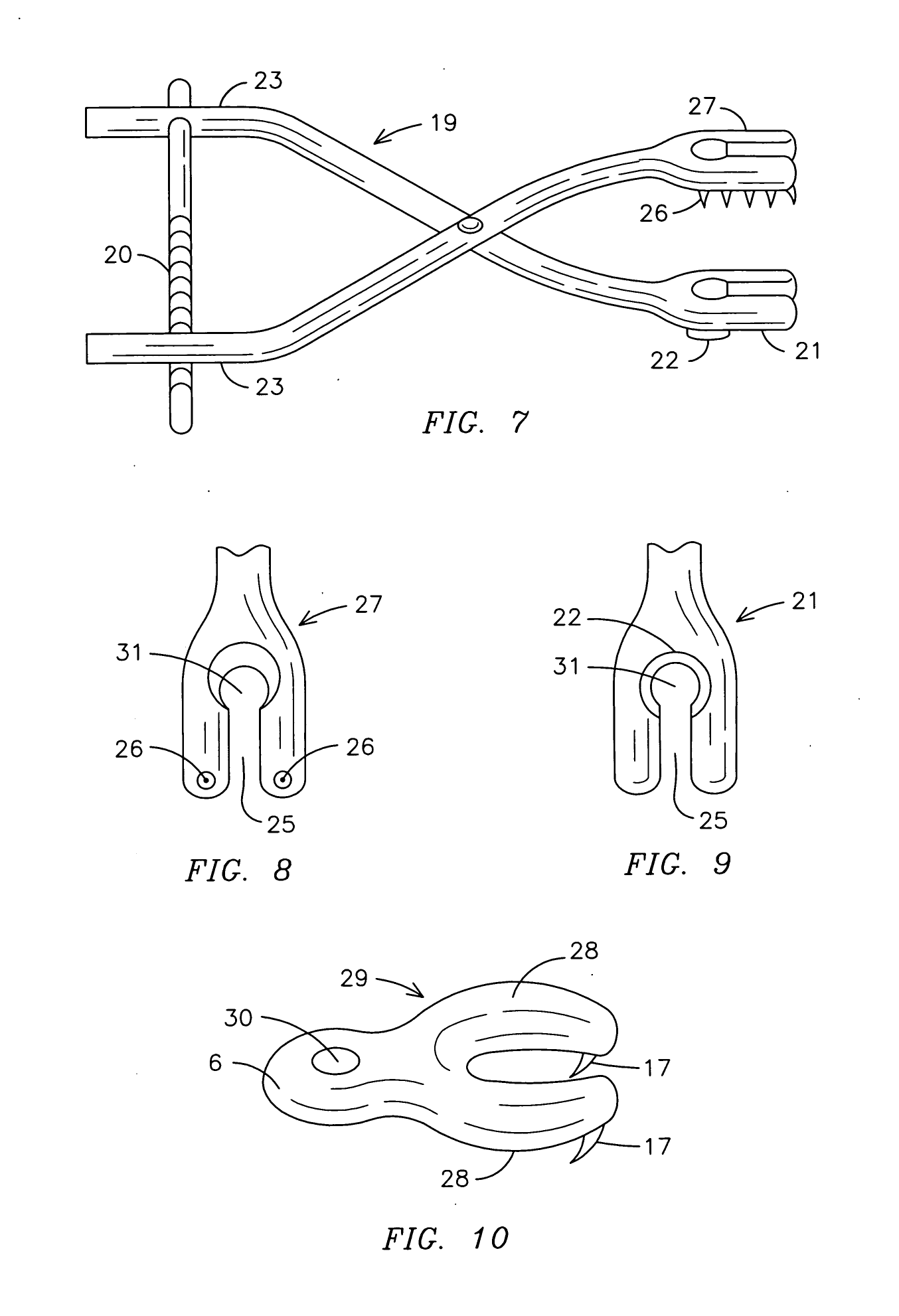

Next, FIG. 7 shows a perspective view of a grasping tool used in the installation of the prosthetic humeral device of the present invention. The grasping tool 19 includes two handles 23, a top circle guide 27 and a bottom circle guide 21. The top circle guide 27 includes at least one spike 26 while the bottom circle guide 21 includes a drill guide projection 22. A locking mechanism 20 allows a user to lock the grasping tool 19 in a predetermined position during use.

FIGS. 8 and 9 show varying views the circle guides 27 and 21 of the grasping tool 19. Each circle guides 27 and 21 includes a slot 25 having a crook 31 that is utilized during the insertion into the prosthetic humeral device 1.

Finally, FIG. 10 shows a perspective view of a second embodiment of a fastening means of the present invention. The fastening means 29 may include a star-shaped washer 6 as shown in FIGS. 1, 2, 4 and 5. In the alternative, the washer 6 may be in various other shapes, including having at least two prongs 28. Using the dual-pronged washer 6, a surgeon is able to aim where the fixation point is gained. Similar to the star-shaped washer 6, the dual-pronged washer 6 is convex and includes projections 17 located on the underside of the washer 6.

To use the present invention, a surgeon performs substantially the same steps traditionally used in current hemi-shoulder replacements. That is, a surgeon first makes an incision into the shoulder and mobilize the chunks of bone that have become broken. Then, the surgeon removes the head, greater and lesser tuberosities from the humerus bone. He or she then drills down into the canal of the humerus to create an area for the insertion of a prosthetic humeral device. The surgeon then inserts the prosthetic humeral device into the canal.

Then however, rather than inserting approximately seven to nine sutures to affix the tendons, the greater tuberosity and the lesser tuberosity to the holes on the fins of the prosthetic humeral device, the surgeon utilizes the grasping tool 19 to firmly grasp and hold each tuberosity 7 and 4 is within the bottom circle guide 21 and top circle guide 27. Then, the surgeon pulls the greater tuberosity 7 and the lesser tuberosity 4 to a predetermined desired level and height in relation to the prosthetic humeral device 1. The drill guide projection 22 located on the grasping tool 19 is then inserted into the corresponding guide hole 11 located on the prosthetic humeral device 1. Using the slots 25 of the grasping tool 19 as a guide, a drill is placed flush against the crook 31 of the grasping tool 19 and a hole is drilled through the tuberosity 7 and 4. A screw 16 that is fed through the hole for the screw 30 in the washer 6 is then inserted through the tuberosity 7 and 4 into the guide hole 11 and is partially secured within the guide hole 11. Thus, the tuberosity 7 and 4 is now sandwiched between the guide hole 11 and the washer 6. Because the grasping tool includes slots 25, the grasping tool 19 is then easily removed from the insertion point of the fastening means 29 and the screw 16 is tightened.

In addition and for added security, the lateral suture holes 12 and medial suture holes 13 may be used to secure the greater and lesser tuberosities 7 and 4 to the prosthetic humeral device 1.

Moreover, because the surgeon can control the exact level at which the greater tuberosity 7 is fixed to the prosthetic humeral device 1 and the screw 16 and washer 6 means for fastening provides a stronger attachment when compared to traditional sutures, the present invention provides a more precise, controlled and stronger tuberosity to bone attachment. In addition, because the screw 16 is compressed, bone ingrowth of the tuberosities 4 and 7 is promoted.

The use of the present invention will allow a surgeon to reduce the amount of time required to perform a hemi-shoulder replacement procedure by at least half.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A prosthetic humeral device comprising:
an elongated stem and a humeral head;
said elongated stem having at least two internally-threaded guide holes located on the elongated stem; and
a means for fastening a greater tuberosity and a lesser tuberosity to said elongated stem using the at least two internally-threaded guide holes.

2. The prosthetic humeral device of claim 1 wherein:
said means for fastening is via a screw and a washer.

3. The prosthetic humeral device of claim 2 wherein:
said washer is star-shaped;
said washer has at least one hole for receiving said screw; and
said washer has an underside.

4. The prosthetic humeral device of claim 2 wherein:
said washer includes at least two prongs;
said washer has at least one hole for receiving said screw; and
said washer has an underside.

5. The prosthetic humeral device of claim 3 wherein:
said washer includes at least one projection located on said underside.

6. The prosthetic humeral device of claim 4 wherein:
said washer includes at least one projection located on said underside.

7. The prosthetic humeral device of claim 1 further comprising:
at least one lateral suture hole located on said humeral head.

8. The prosthetic humeral device of claim 1 further comprising:
at least one medial suture hole located on said humeral head.

9. The prosthetic humeral device of claim 5 further comprising:
at least one lateral suture hole located on said humeral head.

10. The prosthetic humeral device of claim 6 further comprising:
at least one lateral suture hole located on said humeral head.

11. The prosthetic humeral device of claim 7 further comprising:
at least one lateral suture hole located on said humeral head.

12. The prosthetic humeral device of claim 1 further comprising:
a grasping tool that is used in conjunction with said means for fastening a greater tuberosity and a lesser tuberosity to said at least two internally-threaded guide holes.

13. The prosthetic humeral device of claim 12 wherein:
said grasping tool has at least one handle;
said grasping tool having a bottom circle guide and a top circle guide;
a slot located in said bottom circle guide; and
a slot located in said top circle guide.

14. The prosthetic humeral device of claim 13 wherein:
said top circle guide has at least one spike.

15. The prosthetic humeral device of claim 13 wherein:
said bottom circle guide has at least one drill guide projection.

16. The prosthetic humeral device of claim 14 wherein:
said bottom circle guide has at least one drill guide projection.

17. The prosthetic humeral device of claim 12 wherein:
said grasping tool includes a locking mechanism to lock said at least one handle in a predetermined position.

18. A prosthetic humeral device comprising:
an elongated stem and a humeral head;
said elongated stem having at least two internally-threaded guide holes located on the elongated stem;
a means for fastening a greater tuberosity and a lesser tuberosity to said elongated stem using the at least two internally-threaded guide holes;
said means for fastening is via a screw and a washer;
said washer is star-shaped;
said washer has at least one hole for receiving said screw;
said washer has an underside;
said washer includes at least one projection located on said underside;
at least one lateral suture hole located on said elongated stem;
at least one medial suture hole located on said elongated stem;
a grasping tool that is used in conjunction with said means for fastening a greater tuberosity and a lesser tuberosity to said at least two internally-threaded guide holes;
said grasping tool having at least one handle;
said grasping tool having a bottom circle guide and a top circle guide;
a slot located in said bottom circle guide;
a slot located in said top circle guide;
said top circle guide has at least one spike;
said bottom circle guide has at least one drill guide projection; and
said grasping tool includes a locking mechanism to lock said at least one handle in a predetermined position.

19. A method for using a prosthetic humeral device comprising an elongated stem and a humeral head; said elongated stem having at least two internally-threaded guide holes located on the elongated stem; a means for fastening a greater tuberosity and a lesser tuberosity to said elongated stem using the at least two internally-threaded guide holes; and said means for fastening is via a screw and a washer, said method comprising the steps of:
a. grasping said greater tuberosity;
b. drilling a hole through said greater tuberosity;
c. inserting said screw through a hole in said washer;
d. inserting said screw through the drilled hole in the greater tuberosity;
e. inserting said screw into one of said at least two internally-threaded guide holes; and
f. tightening said screw.

20. The method of claim 19 further comprising the steps prior to step f of:
g. grasping said lesser tuberosity;
h. drilling a hole through said lesser tuberosity;
i. inserting said screw through a hole in said washer;
j. inserting said screw through the drilled hole in the lesser tuberosity;
k. inserting said screw into one of said at least two internally-threaded guide holes; and
l. tightening said screw.

21. The method of claim 20 wherein:
said greater tuberosity is grasped using a grasping tool; and
said lesser tuberosity is grasped using a grasping tool.
